(19) United States
(12) Patent Application Publication
(10) Pub. No.: US 2002/0035358 A1
(43) Pub. Date: Mar. 21, 2002

(54) Pulsed electromagnetic field therapy for treatment of corneal disorders and injuries

(76) Inventor: Ming Wang, Nashville, TN (US)

Correspondence Address: Waddey & Patterson, P.C., Bank of America Plaza, Suite 2020, 414 Union Street, Nashville, TN, 37219, US

(21) Appl. No.: 09/853,078

(22) Filed: May 9, 2001

Related U.S. Application Data
(60) Provisional application No. 60/202,780, filed on May 9, 2000.

(51) Int. Cl. A61N 1/00; A61B 18/18; A61B 19/00

(52) U.S. Cl. 606/5; 607/89; 128/898

(57) ABSTRACT

The present invention provides a method for treating corneal ulcers and other corneal conditions with pulsed electromagnetic fields (PEMFs). Specifically, the present invention includes a two part treatment regimen having particular waveforms, intensities, durations, pulse delays, and stepped frequency modulations which maximize the intrinsic healing capacity of cornea.

### BACKGROUND OF THE INVENTION

Cornea is a unique biological tissue. It is normally clear, devoid of vascularization, and comprised of highly organized groups of cells and proteins which make up at least six layers wherein each cell type, protein, and layer performs specialized functions necessary for good vision. The layers of the cornea include: corneal epithelium, basement membrane, Bowman's membrane, stromal lamellae, Descemet's membrane, and the endothelium of the cornea. Injury to the cornea or disease of the cornea can lead to impaired vision and blindness.

Recalcitrant corneal ulcer represents a difficult vision-incapacitating condition for which no effective treatment exists today. These ulcers arise due to a deficiency or an absence of host factors important in wound healing including the lack of tissue perfusion such as an limbal ischemia after chemical burns, decreased corneal innervation and pain sensation (i.e., protective lid closure). Certain examples of corneal ulceration or susceptibility include neurotrophic corneal ulcers after herpetic infections and various dry-eye conditions that compromise the integrity of the ocular surface. Traditional therapies such as lubrication, contact-lens wear, tarsorrhaphy or conjunctival flap are often ineffective. Typically, these patients eventually become blind as a result of corneal melt, perforation or infection. Clearly new and much more effective means of treating these recalcitrant corneal ulcers, and other conditions of the cornea, are urgently needed.

### SUMMARY OF THE INVENTION

The present invention includes methods for treating corneal disorders and injury in a patient with pulsed electromagnetic fields (PEMFs). One aspect of the invention provides a method, comprising: applying a pulsed electromagnetic field (PEMF) to the cornea of the patient. General characteristics of the PEMF treatment for healing cornea include, but are not limited to, a two part treatment regimen having parameters including: waveform, intensity, duration, pulse delay, and stepped frequency modulation as described below.

In certain preferred embodiments, a PEMF is administered to generate or to enhance a wound healing response of a corneal condition. In certain embodiments, a PEMF is administered to stimulate a release of biological factors from a limbal vasculature including, but not limited to: epidermal growth factor (EGF), the transforming growth factor family of growth factors (TGF, including TGFα and TGFβ), and cytokines. In certain embodiments, a PEMF is administered to increase nerve impulse to the cornea. In certain embodiments, a PEMF is administered to induce and accelerate corneal keratocyte activation. In certain embodiments, a PEMF is administered to induce corneal fibroblast transformation, proliferation, and migration. In certain embodiments, a PEMF is administered to induce and accelerate corneal epithelial cell proliferation or migration, especially in the epithelial layer of the cornea. In certain embodiments, a PEMF is administered to induce corneal endothelial cell proliferation and migration. In certain embodiments, a PEMF is administered to enhance the uptake of fluid from the cornea by the corneal endothelial cell layer. In certain other embodiments, a PEMF is administered to diminish the uptake of fluid from the cornea by the corneal endothelial cell layer. In certain embodiments, a PEMF is administered to treat corneal ulcers. In certain embodiments, a PEMF is administered to stimulate tear production from the lacrimal glands of the eye.

### BRIEF DESCRIPTION OF THE DRAWINGS

FIG. 1 is a cross-section of an eye showing the position of the cornea.

FIG. 2 shows a portion of the cornea in cross-section including a depiction of the known layers of specialized corneal tissue.

FIG. 3 is a diagram of a cycle of a pulsed electromagnetic field (PEMF) waveform preferred in a first treatment regimen of a cornea. The X-axis displays time (in this example the unit is a second (s), the drawing is not necessarily to scale). The Y-axis displays magnetic field intensity (in this example the unit is a gauss (G), the drawing is not necessarily to scale). In certain embodiments, the magnetic field intensity is described in units of henry (H). In general, the cycle is repeated for the desired course of treatment as described herein.

FIG. 4 is a diagram of a cycle of a pulsed electromagnetic field (PEMF) waveform preferred in a second treatment regimen of a cornea. The X-axis displays time (in this example the unit is a second (s), the drawing is not necessarily to scale). The Y-axis displays magnetic field intensity (in this example the unit is a gauss (G), the drawing is not necessarily to scale). In certain embodiments, the magnetic field intensity is described in units of henry (H). In general, the cycle is repeated for the desired course of treatment as described herein.

### DETAILED DESCRIPTION OF THE PREFERRED EMBODIMENTS

Cornea is a unique biological tissue. It is normally avascular and is richly innervated by the trigeminal nerve system through a network of subepithelial nerve plexus. In the normal state, the cornea is clear and it may seem to lack substance; however, it is actually a highly organized group of cells and proteins which perform specialized functions necessary for good vision. The primary collagen types found in cornea are 1, 4, and 7. As mentioned, the cornea contains no blood vessels to nourish or protect it against infection. The cornea receives its nourishment from the tears and aqueous humor that fills the chamber behind it. This is unlike any other tissue in the body. The present invention is not bound to mechanism or theory.

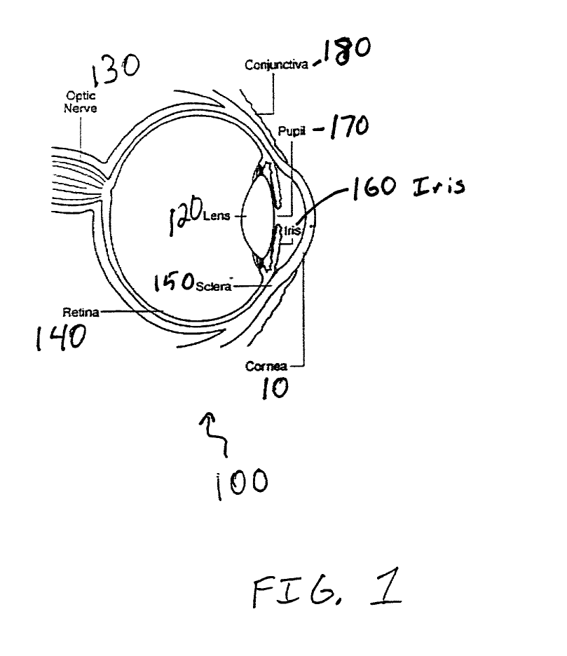

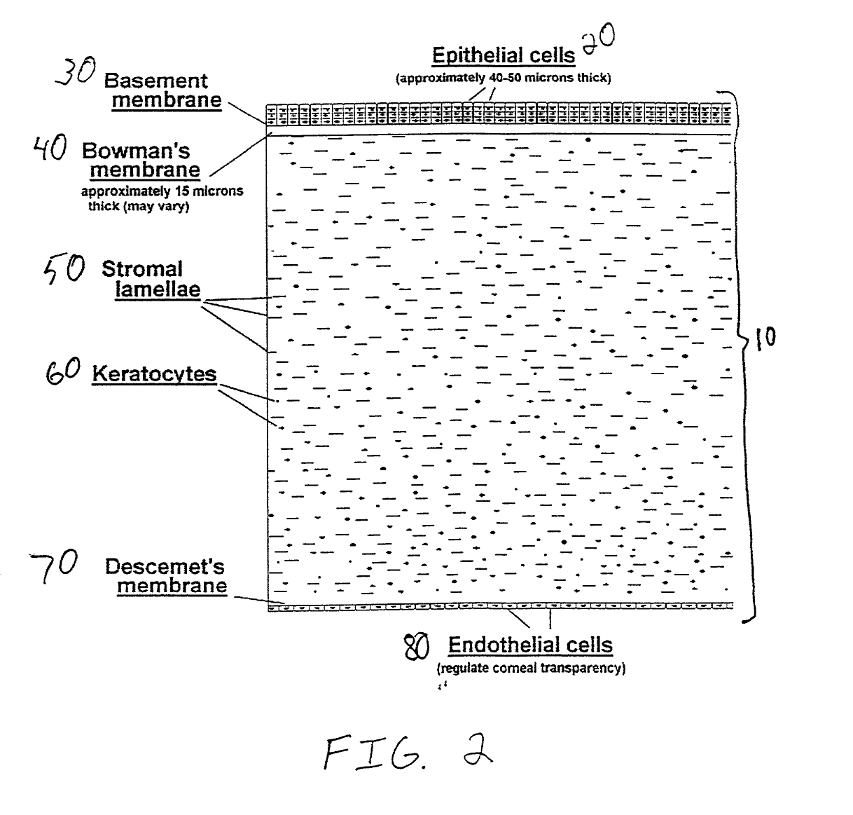

Referring to FIG. 1, the cornea 10 is the transparent tissue that covers the front of the eye 100. Referring to FIG. 2, the structure of the cornea includes the epithelial cell layer 20, the basement membrane 30, the Bowman's membrane 40, the stromal lamellae 50 (which includes keratocytes 60), the Descemet's membrane 70, and the endothelial cell layer 80. Each of these layers can be damaged or diseased and sufficient and proper repair is necessary to promote good eyesight.

The normal cornea 10 is smooth and clear, like glass, but is also strong and durable, like plastic. The cornea 10 provides a physical barrier that shields the inside of the eye from germs, dust, and other harmful matter. The cornea 10 also acts as the outermost lens of the eye 100. When light strikes the cornea 10, it refracts the incoming light onto the crystalline lens 120. The lens then focuses the light onto the retina 140, the paper-thin tissue at the back of the eye 100 that starts the translation of light into vision.

Although much thinner than the lens 120, the cornea 10 provides about 65 percent of the eye's power to refract light. The cornea 10 must remain transparent to refract light properly, and the presence of even the tiniest blood vessels can interfere with this process. To see well, each layer of the cornea 10 (including layers 20, 30, 40, 50, 60, 70, and 80 shown in FIG. 2) must be essentially free of cloudy or opaque areas. Vision is reduced with increasing opacity of the cornea 10 including opacity causes by blood vessel infiltration.

When the cornea 10 is injured, the healing process depends on many factors such as an adequate wound-healing response, a sufficient degree of innervation, and a perfusion of biological factors from limbal vasculature, pre-corneal tear films and the aqueous humor. Delayed wound healing in the cornea occurs in various conditions and may lead to impaired vision. Persistent corneal ulcers occur in instances such as poor tissue perfusion as in chemical injuries, neurotrophic ulcers (for example, due to herpetic infections, strokes or tumor), exposure keratopathy, and various dry-eye conditions that desiccate the ocular surface (e.g., collagen-vascular diseases, conjunctival scarring disorders, vitamin A deficiency, induced by certain medications, and lacrimal gland diseases).

In one example, recalcitrant corneal ulcers may not heal due to a compromised wound-healing response of the host. In order for a wound to close in a normal host, there must be a sufficient amount of stromal 50 wound-healing response involving interaction between the stroma 50 and epithelium 20 containing layers. This includes the activation and migration of the overlying epithelial cells 20, leading to wound closure. In a compromised host, however, the stromal wound healing response is down-regulated and is insufficient to induce the amount of stroma-epithelium (50-20) dialog necessary for wound closure. The PEMF treatments described in the present invention accelerate the wound healing process in the cornea 10.

In another example, the endothelial layer 80 is primarily responsible for pumping fluid into and out of the cornea 10 since the stromal layer 50 does not include blood vessels, at least in the normal state. Any imbalance in the flow of this fluid can damage the cornea 10 and lead to permanent vision impairment including blindness. The PEMF treatments described in the present invention are believed to modulate the fluid pumping action of corneal endothelial cells leading to a proper balance in corneal fluid content.

In the present invention, conditions of the cornea (e.g., corneal diseases and injury) are treated using a pulsed electromagnetic field (PEMF). Without being bound to mechanism or theory, PEMF may treat corneal conditions by augmenting the endogenous tissue reserve for healing. In recent years there has been progress in both the animal experimentation and the clinical use of biomagnetic therapy. A specific form of such therapy, PEMF, has been shown to be effective in treating difficult bone fractures, burns, osteoarthritis, delayed wound healing due to poor vascular perfusion, and soft-tissue and vascular injuries (see references provided below, incorporated herein by reference). In general, each of these therapies is the believed to heal, in part, through the enhancement of vascular performance or vascularization by PEMF treatment. However, the cornea is unique compared to these tissues. For example, the cornea is not vascularized. One of ordinary skill in the art would predict that enhancing the vascularization of the cornea would be counterproductive to the treatment of corneal damage or disease. In fact, the treatment of the cornea or injuries or conditions of the cornea with PEMF is not disclosed in the prior art. Although not bound to this or any mechanism or theory, it is nevertheless, an unexpected and surprising discovery of the present invention that treatment with PEMF is a therapy for enhancing repair of corneal injury and disease.

Generally, any PEMF instrumentation may be used in conjunction with the present invention and numerous PEMF instruments are commercially available. Preferred instruments include those made by Alpha Electronics (GmbH, Hamburg, Germany) and include the Alphatron 4100 Magnetic Field Generator (controller) and the 500 mm coil Magnetic Field Applicator (coil). Alternatively a 315 mm Field Coil or a 700×450 mm MF Flat Applicator may be used. In certain embodiments, the coil may be combined with a multi-phase generator. Multi-phase generators are available from Alpha Electronics, also. At the time of filing, over 300,000 patients have been treated for bone fractures and osteoarthritis worldwide with these instruments and with remarkable efficacy. However, the present invention describes the first treatment of a condition of the cornea with PEMF.

Several specific types of PEMF instrumentation are described in U.S. Pat. Nos. 5,951,459 and 5,997,464 each to Blackwell and each incorporated herein by reference. Additional specific PEMF equipment and methods for use are described in U.S. Pat. No. 5,908,444 to Azure, U.S. Pat. No. 5,269,745 to Liboff et al., and U.S. Pat. No. 4,793,325 to Cadossi et al., each patent incorporated herein by reference.

The PEMF is easily administered to the cornea. One simply places the subject in relation to the field coils in order to deliver defined PEMF energies to the cornea. For example, a subject may lay with his or her head within a cylindrical coil sized to accommodate a child, adolescent, or adult human head. Animals, such as, but not limited to: livestock, farm animals, pets, show animals, horses, dogs, cats, birds, etc. are also within the spirit and scope of the present invention and can be treated according to the principles of the present invention and in light of the present disclosure. In another example, a PEMF generating device can be fashioned according to knowledge in the art in a probe shape (i.e., finger shaped) to administer a given PEMF inductance when placed in defined positions relative to the cornea.

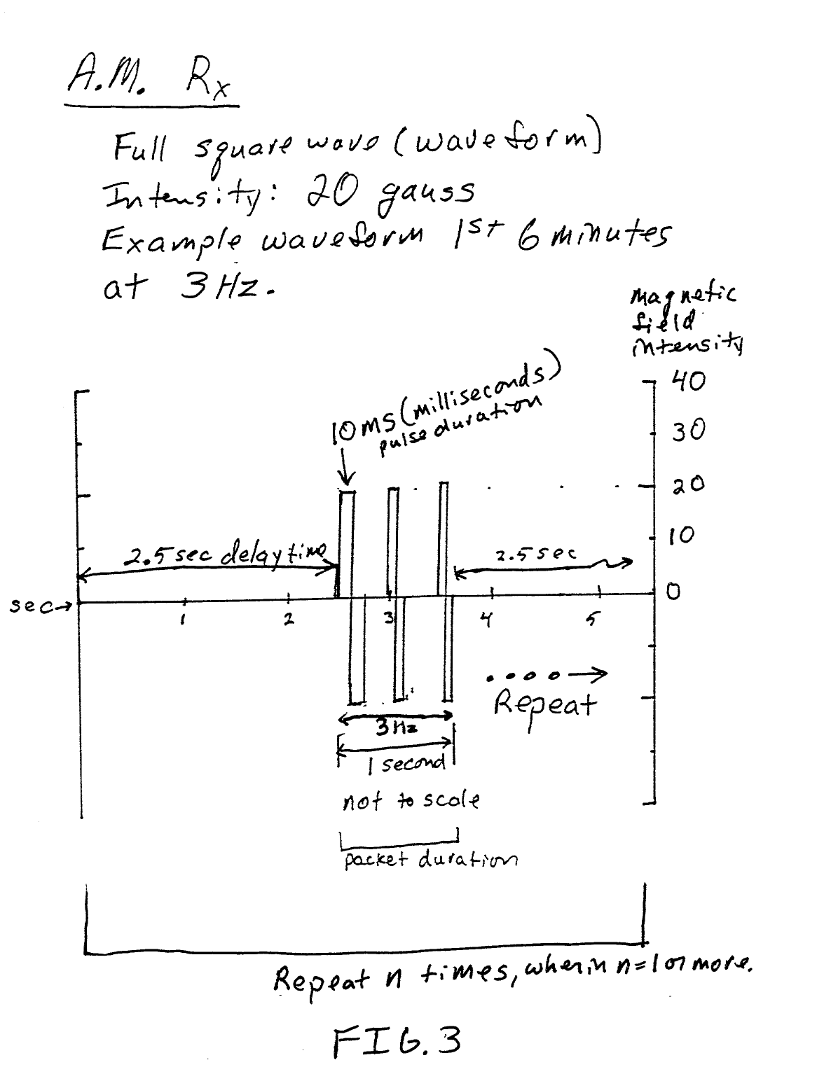

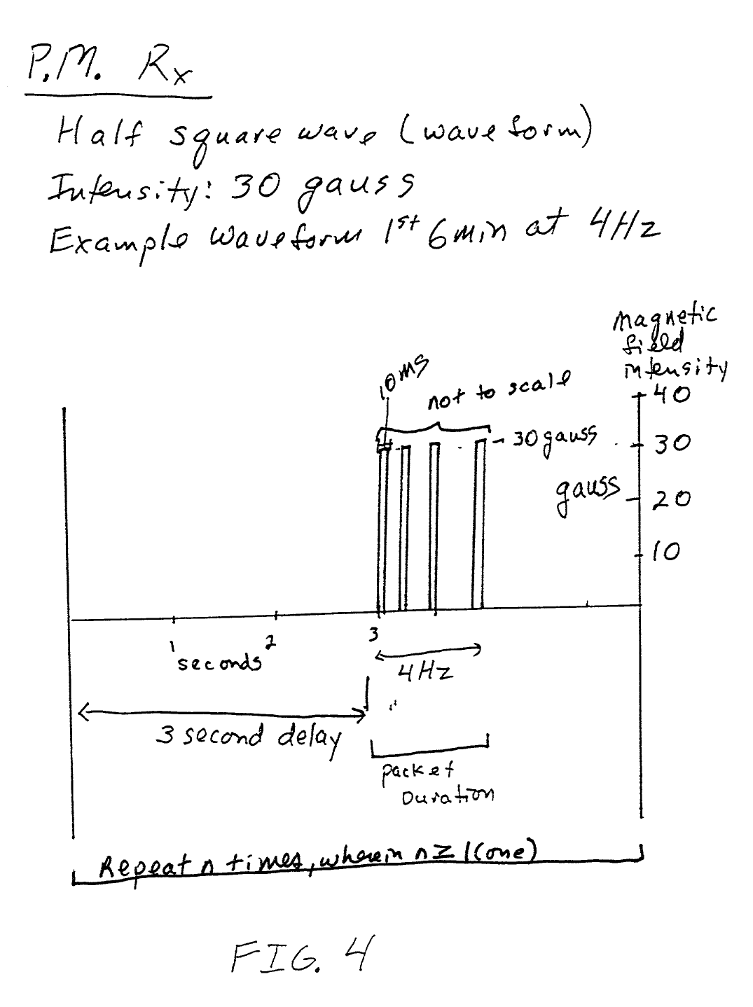

One aspect of the present invention is that the optimal PEMF parameters for accelerating the corneal ulcer and other corneal conditions were found to be different than the parameters previously used for enhancing broken bones and wounds in soft tissue. Thus, one embodiment of the present invention provides a method for treating a condition of the cornea, comprising: a treatment regimen including a plurality of wave packets having a packet duration, a packet frequency, a pulse intensity, a delay time between packets, and a treatment duration (see, for example, FIGS. 3 and 4). The preferred packet duration is approximately 1 second. It is preferred that a wave packet includes one or more electromagnetic pulses having an electromagnetic pulse duration. In certain embodiments, the electromagnetic pulse comprises a full square wave pulse. A preferred electromagnetic pulse duration is approximately 10 milliseconds (ms) and 20 milliseconds in certain other embodiments. In certain preferred embodiments, the preferred delay time (between packets, for example) is approximately 2.5 seconds. In certain preferred embodiments, the pulse intensity is approximately 20 gauss. In certain preferred embodiments, the frequency is changed over time. In certain preferred embodiments, the treatment duration is approximately 30 minutes total and is divided up into 5, approximately 6 minute blocks, wherein the frequency is 3 Hz for a first 6 minute block, 5 Hz for the second six minute block, 7 Hz for the third 6 minute block, 9 Hz for the fourth 6 minute block, and 11 Hz for the fifth 6 minute block.

The number of pulses (electromagnetic pulses) in a packet is generally determined by the packet frequency and the packet duration. For example, as used herein, a 3 Hz frequency refers to a series of 3 electromagnetic pulses over a one second period of packet duration. In another example, as used herein, an 8 Hz frequency refers to a series of 8 electromagnetic pulses over a one second period of packet duration.

Shown in FIG. 3 is a pictorial description of a partial PEMF waveform group preferred in a first treatment regimen which preferably comprises five, six minute periods and is preferably administered in the morning. The waveform is a full square wave with an intensity of approximately 20 gauss. One cycle of the first six minute period is shown wherein the frequency is 3 Hz. During the cycle 3 full square wave pulses of EMF are administered over a 1 second time period (3 Hz) (a batch of 3 pulses), wherein the duration of each half of the full square wave pulse is 10 milliseconds (ms). Following the three pulses (3 Hz) a 2.5 second delay period is maintained. The pulses and 2.5 second delay are repeated for the six minutes of the first period. In the second 6 minute period (not shown) 5 pulses are administered in the one second time period (5 Hz) with a 2.5 second delay between each batch of pulses. In the third 6 minute period (not shown) 7 pulses are administered in the one second time period (7 Hz) with a 2.5 second delay between each batch of pulses. In the fourth 6 minute period (not shown) 9 pulses are administered in the one second time period (9 Hz) with a 2.5 second delay between each batch of pulses. In the fifth 6 minute period (not shown) 11 pulses are administered in the one second time period (11 Hz) with a 2.5 second delay between each batch of pulses.

In certain preferred embodiments, the method further comprises a second treatment regimen. The second treatment regimen includes a plurality of wave packets having a packet duration, a packet frequency, a pulse intensity, a delay time between packets, and a second treatment duration (see FIG. 4). The preferred packet duration is 1 second. It is preferred that a wave packet includes one or more electromagnetic pulses having an electromagnetic pulse duration. In certain embodiments of this second treatment regimen, the electromagnetic pulse comprises a half square wave pulse. A preferred electromagnetic pulse duration is approximately 10 milliseconds (ms). In certain preferred embodiments, the preferred delay time is approximately 3.0 seconds. In certain preferred embodiments, the pulse intensity is approximately 30 gauss. In certain preferred embodiments, the frequency is changed over time. In certain preferred embodiments, the treatment duration is approximately 30 minutes total and is divided up into 5, approximately 6 minute blocks, wherein the frequency is 4 Hz for a first 6 minute block, 6 Hz for the second six minute block, 8 Hz for the third 6 minute block, 10 Hz for the fourth 6 minute block, and 12 Hz for the fifth 6 minute block.

Shown in FIG. 4 is a pictorial representation of a partial therapeutic PEMF waveform group preferred in the second treatment regimen which preferably comprises five, six minute periods and is preferably administered in the afternoon. The waveform is a half square wave with an intensity of approximately 30 gauss. One cycle of the first six minute period is shown wherein the frequency is 4 Hz. During the cycle 4 half square wave pulses of EMF are administered over a 1 second time period (4 Hz) (a batch of 4 pulses), wherein the duration of each half of the half square wave pulse is 10 milliseconds (ms). Following the four pulses (4 Hz) a 3.0 second delay period is maintained. The pulses and 3.0 second delay are repeated for the six minutes of the first period. In the second 6 minute period (not shown) 6 pulses are administered in the one second time period (6 Hz) with a 3.0 second delay between each batch of pulses. In the third 6 minute period (not shown) 8 pulses are administered in the one second time period (8 Hz) with a 3.0 second delay between each batch of pulses. In the fourth 6 minute period (not shown) 10 pulses are administered in the one second time period (10 Hz) with a 3.0 second delay between each batch of pulses. In the fifth 6 minute period (not shown) 12 pulses are administered in the one second time period (12 Hz) with a 3.0 second delay between each batch of pulses.

In preferred embodiments, the method further comprises a treatment regimen separation time that divides the first and the second treatments regimens of the method. A preferred regimen separation time is approximately 5 hours from the beginning of the first regimen to the beginning of the second regimen.

In certain preferred embodiments, the method further comprises repeating the first and the second treatment regimen daily for a course of treatment. A preferred course of treatment is approximately 9 consecutive days.

In aspects of the present invention, the inventor has discovered that a combination treatment wherein two separate administrations given daily is optimal for treating corneal conditions. It is preferred that the first and the second administration are separated by approximately 5 hours; however, this time period can be more or less than 5 hours. It is preferred that the first treatment regimen include a full square wave PEMF of approximately 20 gauss and with a pulse delay of approximately 2.5 seconds. It is also preferred that the frequency of the PEMF be varied. A preferred arrangement of frequency variation includes a step arrangement from 3 Hz to 11 Hz in steps of 2 Hz, wherein each step is performed for a period of approximately 6 minutes. It is preferred that the second treatment regimen include a half square wave PEMF of approximately 30 gauss and with a pulse delay of approximately 3.0 seconds. It is again preferred that the frequency of the PEMF be varied. A preferred arrangement of frequency variation includes a step arrangement from 4 Hz to 12 Hz in steps of 2 Hz, wherein each step is performed for a period of approximately 6 minutes. The frequencies are approximate.

Certain embodiments of the present invention provide a method of treating a corneal condition (including injury or disease), comprising: administering a therapeutically effective dose or regimen of a pulsed electromagnetic field to the cornea. In certain embodiments, the condition of the cornea is a ulcer or corneal ulcer. In certain embodiments, a therapeutically effective dose or regimen is one in which corneal healing is accelerated. For example, the repair or healing of the cornea is accelerated in comparison to one or more controls which are under similar circumstances with the exception that the PEMF is not turned activated. Thus, the control(s,) and even the administrator of the therapy, may fully expect that the control(s) have been treated; however, there has been a predetermined interruption of power to the generator, for example.

In certain embodiments, the PEMF includes a packet duration having discrete signals of magnetic field intensity (e.g., in units of henry (H)) or induction (e.g., in units of gauss (G)). In general one or more packet duration events are applied (see, for example FIGS. 3 and 4). In certain embodiments, the packet duration can vary considerably, including, but not limited to: approximately 0.1 seconds (s), 0.2 s, 0.3 s, 0.4 s, 0.5 s, 0.6 s, 0.7 s, 0.8 s, 0.9 s, 1.0 s, 1.1 s, 1.2 s, 1.3 s, 1.4 s, 1.5 s, 1.6 s, 1.7 s, 1.8 s, 1.9 s, 2.0 s, 2.1-3.0 s, 3.1-4.0 s, and 4.1-5 s, and 0.05 to 10 s. A preferred packet duration is approximately 1.0 seconds. In certain embodiments, the discrete signals or pulses within the packet duration can vary considerably, including but not limited to: approximately 1 millisecond (ms), 2 ms, 3 ms, 4 ms, 5 ms, 6 ms, 7 ms, 8 ms, 9 ms, 10 ms, 11 ms 12 ms, 13 ms, 14 ms, 15 ms, 16 ms, 17 ms, 18 ms, 19 ms, 20 ms, 21 ms, 22 ms, 23 ms, 24 ms, 25 ms, 26-30 ms, 31-40 ms, 41-50 ms, 51-100 ms, and 0.5 to 200 ms. In certain embodiments, the waveform of the pulses can vary considerably including, but not limited to: square wave, ½ square wave, sine wave, ½ sine wave, cosine wave, ½ cosine wave, and combinations thereof. An example of a combination would be mixing full square waves with ½ square waveforms within a packet. In certain embodiments, the intensity of the pulse can vary considerably including, but not limited to: approximately 1-1000, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51-100, 101-200, and 201-500 (the units for these values can be H in certain embodiments and G in certain embodiments). In certain embodiments, the delay time can vary considerably, including but not limited to: approximately 0.1-1 s, 1.1-2.0s, 2.1 s, 2.2 s, 2.3 s, 2.4 s, 2.5 s, 2.6 s, 2.7 s, 2.8 s, 2.9 s, 3.0 s, 2.1 s to 3.0 s, 3.1-4.0 s, 4.1-10 s.

Without being bound to mechanism or theory, it is proposed that PEMF results in improvements in conditions of the cornea through a variety of effect including, but not limited to: induction and acceleration of corneal keratocyte activation and migration; induction of corneal fibroblast transformation, proliferation, and migration; induction and acceleration of corneal epithelial cell proliferation or migration in the epithelial layer of the cornea; induction of corneal endothelial cell proliferation and migration; enhanced regulation of fluid pumping in and out of the corneal (especially the stromal lamellae) by the corneal endothelial cell layer; increased corneal nerve stimulation, and through the induction of tear production from the lacrimal glands of the eye. Additionally, up-regulation of stromal cellular activities is believed to result in an increased stromal-epithelium dialog that leads to the activation and accelerated migration of overlying corneal epithelium and corneal wound closure.

Specific cellular and molecular responses that may mediate biological responses described in the present invention include: an up-regulation of DNA synthesis and mRNA transcription of protein factors involved in wound healing; reduction of the doubling time of fibroblasts and endothelial cells in cell cultures; induction of fibroblast differentiation in cell cultures; augmentation of collagen synthesis, angiogenesis, and bacteriostasis in wound healing; and regulation of membrane transport, receptor expression and signal transduction pathways.

Certain conditions, disorders, and diseases of the cornea that can be treated by way of the present invention, include, but are not limited to: corneal injury, corneal ulcer, burns, conjunctivitis, abrasions, edema, opacity, transplant (i.e., healing following transplant), dry eyes, infections in general, herpes simples, herpes zoster (shingles), giant papillary conjunctivitis, keratoconus, pterygia and pingueculae, and damage due to laser surgery (e.g., laser vision correction including phototherapeutic keratectomy, LASICS, etc.).

Although these preferred embodiments of the invention are described, one with skill in the art will realize that variations can be made which yield similar results in terms of healing of corneal ulcers or other corneal conditions.

### EXAMPLES

### Example 1

A double masked placebo-controlled study was conducted to measure the therapeutic effect of a pulsed electromagnetic field (PEMF) for increasing the rate of epithelial healing in rabbit corneal ulcers. Twelve rabbits received iatro-genic corneal ulcers secondary to surgical and chemical debridement of the corneal epithelium and limbal stem cells. The experimental group (6 rabbits, 12 eyes) received PEMF therapy 2-3 times daily for 19 days, while the control group (6 rabbits, 12 eyes) received sham treatments. The amount of epithelial healing was measured daily by slit lamp biomicroscopy of fluorescein stained wounds in terms of the size of corneal the ulcer in square mm (mm²) as is known in the art.

Wound healing in the rabbit corneal ulcers was statistically significantly different in the PEMF treated group compared to the sham treated group. The time required for 50% corneal healing was 8.6±0.3 days for the treated group, and 11.0±0.4 days for the control group (p,0.01). The time required for 75% corneal healing was 11.0±0.4 days for the treated group, and 14.5±0.3 days for the control group (p<0.01). The percent corneal healing at the 50% duration-time was 57±10% for the treated group, and 28±12% for the control group (p<0.01). These data demonstrate that PEMF therapy increases the rate of wound healing of rabbit corneal wounds. These data also demonstrate that PEMF therapy provides a novel therapy for recalcitrant corneal ulcers, as shown in this rabbit model system.

The PEMF instrument used was the Alphatron 4100 Magnetic Field Generator which includes a 500-mm coil Magnetic Field Applicator. The instrument is made by Alpha Electronics (GmbH, Hamburg, Germany). Each rabbit was housed in the center of the 500 mm coil applicator during the treatment. The treatment group of rabbits received treatment twice a day, for 9 consecutive days (postoperative day 1 to day 9). The first daily treatment, referred to as the A.M. treatment, was administered at approximately 9:00 A.M. The second daily treatment, referred to as the P.M. treatment, was administered at approximately 2:00 P.M.

The following treatment protocol was discovered by the inventor to be the optimal A.M. treatment prescription for corneal ulcer healing. Packets of energy including full square wave pulses were administered at 20 gauss pulse intensity with a packet delay of 2.5 seconds between packets, a pulse duration of 10 ms and the following packet frequency profile: 6 minutes at 3 Hz, followed by 6 minutes at 5 Hz, 6 minutes at 7 Hz, 6 minutes at 9 Hz, and 6 minutes at 11 Hz. Thus, the packet was followed by the delay which was repeated for 6 minutes at each packet frequency. Also, by way of explanation, a 5 Hz packet frequency means that 5 pulses occur during one second of packet duration, in this case one second; so, there are 5 pulses per packet at 5 Hz.

The following treatment protocol was discovered by the inventor to be the optimal P.M. treatment prescription for corneal ulcer healing. Packets of energy including half-square wave pulses were administered at 30 gauss pulse intensity with a packet delay of 3.0 seconds, a pulse duration of 10 ms, and the following frequency profile: 6 minutes at 4 Hz, followed by 6 minutes at 6 Hz, 6 minutes at 8 Hz, 6 minutes at 10 Hz, and 6 minutes at 12 Hz.

### Example 2

A prospective and randomized study is conducted to measure the effect of PEMF treatment on the rate of corneal epithelial wound closure in a rabbit model of the condition. Rabbit is an excellent animal model for studying corneal wound healing because of the resemblance or rabbit cornea to human cornea histologically, the large size of the rabbit cornea makes it readily amendable for surgical manipulation, and a large volume of established literature related to corneal wound healing in rabbits is known (Hanna et al. (1989) ARCH OPHTHAL 107:895-901 and Tuft et al. (1987) LASERS IN OPHTHAL 1:177-183; each article incorporated herein by reference).

An accurate and reproducible corneal wound, is produced in the cornea using the VISX STAR™ excimer laser at the Vanderbilt Laser Vision Center™. The excimer laser, with its output at 193 nm, has been proven to be effective in creating consistent corneal wound in rabbits (Hanna (1989) supra, and Wang et al. (1997) INVET OPHTHAL VIS SCI 38:S405, incorporated herein by reference). The output wavelength of this laser is 193 nm, the frequency is 5 Hz, and the fluence is 160 mJ/cm². The ablation parameters will include the following (not limiting): 6 mm diameter, 120 μm depth, and using the transepithelial approach and the phototherapeutic keratectomy mode (PTK) of the instrumentation.

### Example 3

After laser ablation, rabbits will undergo PEMF treatment and the rate of epithelial closure of the corneal ulcers will be measured daily in the postoperative days and compared between the PEMF-treated and the control groups (see the following Examples). A battery of testing rounds will be performed to determine the optimal ranges for stimulating wound healing, specific cellular responses, and other endpoints. PEMF parameters to be tested include, but are not limited to: pulse frequency, amplitude, waveforms, and timing and duration of treatment. Waveforms that will be tested include, but are not limited to: ½ sine wave, full sine wave, ½ square wave, and full square wave. Each parameter including the waveform will be tested using a 315 mm PEMF Coil, a 700×450 mm PEMF MF Flat Applicator, and a 500 nm PEMF Coil with the Alphatron 4100 Magnetic Field Generator by Alpha Electronics.

### Example 4

Healing of laser induced corneal wounds (as described in Example 2). In one experiment 36 rabbits will be divided into four groups. The experiment will adhere to the Vanderbilt University Institutional Review Board (IRB) Protocol (incorporated herein by reference) for using animals in research. The rabbits will receive adequate anesthesia preoperatively and antibiotic topical medications postoperatively. All rabbits will be coded and the examiner will be blind to this code in the postoperative evaluation of wound closure rate.

A total of 8 rabbits will be used for a testing group #1 for the 315 mm PEMF coil. After bilateral excimer laser ablation, supra, the 8 rabbits will be evenly subdivided into four groups (2 rabbits each), each group will receive treatment with one of four PEMF waveforms (½ sine, full sine, ½ square, full square). The 315 mm Coil PEMF system has a concentric coil configuration with a cross-sectional diameter of 315 mm. At the coil cross-sectional XY plane, the maximal B field at the rim is 120 gauss with a corresponding center field of 60 gauss. The rabbits will be placed in the center of the coil with an effective average B field of 80 gauss. The PEMF treatment will be performed twice a day (30 minutes at 10 Hz) in the postoperative period (following laser ablation).

A total of 8 rabbits will be used for a testing group #2 for the 700×450 mm PEMF system. The protocol will be identical to that of 315 mm system with the exception that the 700×450 mm system can simultaneously house and treat 4 rabbits with an average effective B field strength of 40 gauss.

A total of 10 rabbits will be used for a testing group #3. The 10 rabbits in this group will receive bilateral excimer laser ablation, and 5 rabbits will receive the 315 mm treatment using the optimized treatment parameters found in the previous corresponding testing run. In this study the sample size NM=20, which is necessary to achieve sufficient degree of statistical significance for comparison between the 315 mm PEMF-treated and the control rabbits.

A total of 10 rabbits will be used for a testing group #4. This is the treatment group for the 700×450 mm PEMF system using the identical protocol of the 315 mm system for group #3.

The corneal epithelial closure rate (mm²/day) is measured by the size of fluorescein stain. The following postoperative measurement protocol will be used (the numbers refer to postoperative days): 1, 2, 3, 4, 5, 6, and 7. These experiments with be performed for the 315 mm PEMF Coil and control (sham treated) and the 700×450 mm PEMF Coil and control (sham treated).

### Example 5

Example 4 is repeated using the Alphatron 4100 Magnetic Field Generator and the 500 mm PEMF Coil.

### Example 6

The previous examples are performed wherein on each postoperative day, a randomly chosen rabbit will be sacrificed and globe enucleated for histologic studies. One half of each cornea will be sectioned and H & E stain performed to determine the extent of cellular inflammatory response, and cellular activation and migration. The second half of each cornea will be used for the characterization of the keratocyte apoptosis study using the TUNEL technique (Wang, supra). In previous studies the inventor has demonstrated down-regulation of wound healing responses including inflammation and keratocyte apoptosis using amniotic membrane transplantation (Wang, supra). The inventor has discovered that, in the present invention, PEMF treatment yields the opposite effect, namely, an enhanced wound healing response.

### Example 7

A clinical trial will be conducted with a total of one-hundred patients divided into a PEMF treatment group (50 patients) and a control group (fifty patients). All patients will be examined by one or more independent ophthalmologist who will measure corneal injury by methods known in the art. A value scale of corneal damage from 1 to 10 will be assigned to each patient. The patients will be divided into the PEMF treatment group and the control group such that the summation of the value scale of corneal damage for each group is approximately equal. The person assigning the groupings will be blind to the patient identities and cases.

Both the PEMF and the control group will receive standard care for corneal injury as is known in the art and as directed by their treating physician. In addition, the PEMF treatment group will receive PEMF treatments according to the following protocol. The control group will be treated in the same manner with the PEMF protocol except that the field coil will not activated unknown to the patients.

The PEMF instrument will be an Alphatron 4100 Magnetic Field Generator and a 500-mm coil Magnetic Field Applicator. The cornea of each patient will be placed in the center of the 500 mm coil applicator during the treatment. PEMF treatment will be administered twice a day, for 9 consecutive days (following the scoring of the corneal injury). The treatments used will be the A.M. and P.M. treatments described in Example 1.

At the end of the nine day period, each patient will be examined again (by physicians that do not know which treatment protocol was received) and the remaining amount of corneal injury will be measured according to the same scale. The relative improvement in corneal healing will be determined statistically between the PEMF treatment and the control groups.

All references, patents (including U.S. and otherwise), articles, and the like referred to herein are hereby incorporated herein by reference in their entirety, including the following references.

Reference
Andrew, C., et al. Pulsing electromagnetic field treatment in ununited fractures and failed arthrodes. JAMA.247;1982.
Assimacopulos, D. Wound healing promotion by the use of negative electric current. Am Surg 34:423-431;1968.
Barker, AT., et al. Pulsed magnetic field therapy for tibial non-union : Lancet May994;1984.
Bassett, C.A.L., et al. Pulsing electromagnetic fields treatment in ununited fractures and failed arthrodeses. JAMA24:623-628;1982.
Bassett, C.A.L., Low-energy pulsing electromagnetic fields modify biomedical processes Bioessays 6:36-42;1987.
Bassett, C.A.L., Treatment of ununited tibial diaphyseal fractures with pulsing electromagnetic fields. J Bone & Joint Surg 63A:511-523;1981
Beers, G.J. Biological effects of week electromagnetic fields from 0 Hz to 200 Hz: a survey of the literature with special emphasis on
possible magnetic resonance effects. Man Reson Imaging 7:309-331;1989.
Einfelds, H. First experiences with magnetic field therapy in connection with ulera crurum. Dept. Physical Therapy of the General
Hospital St. Georg. Hamburg 7. 1996.
Erdman, W.J. Peripheral blood flow measurements during application of pulsed high frequency currents. Am J Orthop; 1960.
Foulds, L.S., et al. Human skin battery potentials and their possible role in wound healing. Br. J Dermatol 109:515-522;1983.
Goodman, R., et al. Some biological effects electromagnetic fields. Biochemistry and Bioenergetics 15:39-55;1986.
Hanna, K.D., et al. Corneal stroma wound healing in rabbits after 193 cm examiner laser surface ablation. Arch Ophihal 107:895-901;1989.
Lee, S., and Tseog. S.C.G. Amniotic membrane transplantation for persistent epithelial defects with ulceration. Am J Ophihal
123:303-312;1997.
Mulder, G. Treatment of open skin wounds with electric stimulation. Arch Phys Med Rehahil T2-375-377;1991.
Murray, J.C. Farndale R.W. Modulation of collage production in cultured fibroblasts by a low-frequency pulsed magnetic field. Biochim
Biophys Acts. 838:98-105;1985.
Pollack, S.V., et al. Effect of pulsed electrical stimulation on dermal ulcer repair (abs). Bioelectric Repair & Growth Society
Proceeding, Wash. DC., 1988.
Reich, J.D., et al. Electrical stimulation of skin: a review. Int J Dermatol 29:395-400;1990.
Rodemann, H.P., et al. The proliferation of normal and transformed human fibroblasts in vitro is influenced by electromagnetic fields.
Exp Cell Res 182;610-621;1989
Sharp, L.K., Lightwood, R. Stimulation of bone union by externally applied radio-frequency energy. Injury 14:523-530;1983.
Shartard, W.J.W. A double-blind trial of pulsed electromagnetic fields for delayed union of tibial fracture. J of Bone & Joint Surg
British Vol No.3. 1990.
Stiller, M.J., et al. A portable pulsed electomagnetic field (PEMF) device to enhance healing of recalcitrant venous ulcers:
a double-blind, placebo-controlled
clinical trial. Bri J Dermatol 127:147-154;1992.
Trock, D.H., et al. A double-blind trial of the clinical effects of pulsed electromagnetic fields in osteoarthritis. J Rheumatol 1993.
Turt, S., et al. Stroll remodeling following photorefractive keradectomy. Lasers in Ophithal 1:177-183;1987.
Wang, M.X., et al. Corneal haze and keratocyle apoplosis is reduced with amniotic membrane transplantation after excimer laser
ablation in rabbits. Invet Ophthal Vis Sci 38:S405;1997.
Weiss, D.S., et al. Electrical stimulation and wound healing. Arch Dermatol 126:222-225;1990.
Wilson, D.H. Experimental regeneration in peripheral nerve and spinal cord in laboratory animals exposed to a pulsed
electromagnetic field. Paraplegia May:12-19:1976.
Yen-Patton, G.P.A. et al. Endothelial cell response to pulsed electromagnetic fields: stimulation of growth rate and angiogenesis in vitro
J Cell Physiol 134:37-46:1988.
Zaffuto, M.I.S., et.al. Effect of low frequency pulsing electromagnetic fields on skin ulcers of venous origin in humans: a
double-blind study. J Orthopaetid Res 8:276-282:1990.

What is claimed is:

1. A method of treating a cornea in need of treatment for a corneal ulcer, comprising: administering a therapeutically effective regimen of a pulsed electromagnetic field to the cornea.
